(19) United States
(12) Patent Application Publication
(10) Pub. No.: US 2017/0239240 A1
(43) Pub. Date: Aug. 24, 2017

(54) Sustained Release Buprenorphine Microshperes (SRBM) and Methods of Use Thereof

(71) Applicant: BioDelivery Sciences International, Inc., Raleigh, NC (US)

(72) Inventors: Niraj Vasisht, Cary, NC (US); Andrew Finn, Raleigh, NC (US)

(21) Appl. No.: 15/441,197

(22) Filed: Feb. 23, 2017

Related U.S. Application Data
(60) Provisional application No. 62/298,777, filed on Feb. 23, 2016.

(51) Int. Cl. A61K 31/485; A61K 9/00; A61K 9/16

(52) U.S. Cl. CPC A61K 31/485; A61K 9/1635; A61K 9/1647; A61K 9/1617; A61K 9/0019

(57) ABSTRACT

The present invention is directed to a sustained release buprenorphine microsphere (SRBM) formulation capable of delivering buprenorphine, a metabolite, or a prodrug thereof for a duration of about 7 days to about 6 months.

### CROSS-REFERENCE TO RELATED APPLICATIONS

This application claims priority to U.S. Provisional Application No. 62/298,777, filed on Feb. 23, 2016, the entire contents of which are incorporated herein by reference.

### BACKGROUND

Buprenorphine is marketed under several trade names, one of which is Bunavail® for the maintenance treatment of opioid dependence. Buprenorphine HCl has the molecular formula C₂₉H₄₁NO₄ HCl and the molecular weight is 504.10. It is a white or off-white crystalline powder, sparingly soluble in water, freely soluble in methanol, soluble in alcohol, and practically insoluble in cyclohexane. Buprenorphine has been used to treat opioid dependence and pain. There are both tablet formulations, sublingual oral films and buccal film formulations available as well as a 7 day transdermal patch. The oral formulations are approved for once or twice per day administration, but may be administered three times daily in some cases and the transdermal patch frequently does not provide 7 day pain control due to variable pharmacokinetics. There is a need therefore for methods of administering buprenorphine that have short and long term stable release kinetics.

### SUMMARY

Provided herein are methods of treating opioid dependence and/or pain with a sustained release formulation that provides therapeutic levels of a pharmaceutically active agent for up to 30 days or more. In one embodiment, immediately after administration and for the first 12 hours there is not a substantial burst of pharmaceutically active agent released.

Provided herein are methods for treating opioid dependence and/or pain in a subject comprising, administering a therapeutically effective amount of a sustained release buprenorphine microsphere (SRBM) formulation, wherein the formulation comprises buprenorphine and poly(D,L-lactide-co-glycolide) having a MW of 24,000-38,000, wherein the plasma concentration does not exceed 10 ng/ml in the 12 hours after administration. In certain embodiments, the plasma concentration does not exceed 8 ng/ml in the 12 hours after administration. In certain embodiments, the plasma concentration does not exceed 7.5 ng/ml in the 12 hours after administration. In certain embodiments, the plasma concentration does not exceed between about 5 and about 8 ng/ml in the 12 hours after administration.

Provided herein are methods for treating opioid dependence and/or pain in a subject comprising, administering a therapeutically effective amount of a sustained release buprenorphine microsphere (SRBM) formulation, wherein the formulation comprises: buprenorphine and poly(D,L-lactide-co-glycolide) having a MW of 7,000-17,000, wherein the plasma concentration of buprenorphine does not exceed 10 ng/ml in the 12 hours after administration.

Provided herein are methods for treating opioid dependence and/or pain in a subject comprising, administering a therapeutically effective amount of a sustained release buprenorphine microsphere (SRBM) formulation, wherein the SRBM formulation comprises, buprenorphine, poly(D,L-lactide-co-glycolide) having a MW of 24,000-38,000 and poly(D,L-lactide-co-glycolide) having a MW of 7,000-17,000. In one embodiment, plasma concentration of buprenorphine does not exceed 10 ng/ml in the 12 hours after administration.

In one embodiment, the SRBM formulation further comprises polyvinyl alcohol.

In one embodiment, the SRBM formulation further comprises ethyl acetate.

In one embodiment, the pain is chronic pain.

In one embodiment, the pain is chronic pain in an opioid dependent subject.

In one embodiment, the plasma concentration of buprenorphine remains substantially in the therapeutic range for 27 of 31 days. In one embodiment, the plasma concentration of buprenorphine remains substantially in the therapeutic range for greater than 28 days.

In one embodiment, the therapeutic range comprises from about 1 ng/ml to about 8 ng/ml. In one embodiment, the therapeutic range comprises from about 1 ng/ml to about 8 ng/ml for the treatment of opioid dependence. In one embodiment the therapeutic range comprises from about 1 ng/ml to about 5 ng/ml for the treatment of pain. Therapeutic range comprises from about 1 ng/ml to about 8 ng/ml for the treatment of pain in opioid dependent subjects.

In one embodiment, wherein the composition SRBM formulation comprises from about 1 milligrams to about 600 milligrams of buprenorphine.

In one embodiment, the buprenorphine is in the form of a free base or pharmaceutically acceptable salt.

In one embodiment, the SRBM formulation after administration, the plasma concentration of buprenorphine remains substantially in the therapeutic range for 7 days.

In one embodiment, the SRBM formulation after administration, the plasma concentration of buprenorphine remains substantially in the therapeutic range for between 3 and 7 days.

In one embodiment, the method comprises administering the SRBM formulation to the subject once about every 7 days by injection.

In one embodiment, the method comprises administering the SRBM formulation to the subject once about every 30 days by injection.

In one embodiment, the method comprises administering the SRBM formulation to the subject once about every 60 days by injection.

In one embodiment, the method comprises administering the SRBM formulation to the subject once about every 120 days by injection.

In one embodiment, the method comprises administering the SRBM formulation to the subject once about every 180 months by injection.

In one embodiment, the SRBM formulation has a substantially linear release profile that lasts for 30 days after administration to the subject.

In one embodiment, the therapeutically effective dose of SRBM formulation is administered a second time at least about 15 days, 30 days, 45 days, 60 days, 90 days, 120 days or 180 days after administration.

In one embodiment, the second administration will have a burst that is additive to the subject's plasma concentration from a previous administration.

In one embodiment, the burst is less than 10 ng/ml above the subject's plasma concentration at the time of administration of the second dose.

In one embodiment, the burst is less than 9 ng/ml above the subject's plasma concentration at the time of administration of the second dose.

In one embodiment, the burst is less than 8 ng/ml above the subject's plasma concentration at the time of administration of the second dose.

In one embodiment, the burst is less than 7 ng/ml above the subject's plasma concentration at the time of administration of the second dose.

In one embodiment, the burst is less than 6 ng/ml above the subject's plasma concentration at the time of administration of the second dose.

In one embodiment, the burst is from between about 3.5 ng/ml and about 10 ng/ml above the subject's plasma concentration at the time of administration of the second dose.

In one embodiment, the burst is from between about 4 ng/ml to about 10 ng/ml above the subject's plasma concentration at the time of administration of the second dose.

In one embodiment, the administration is a subcutaneous injection with a needle from between about 23 to about 27 gauge.

In one embodiment, the administration is a subcutaneous injection with a 27 gauge needle.

In one embodiment, the administration is a subcutaneous injection with a 26 gauge needle.

In one embodiment, the administration is a subcutaneous injection with a 25 gauge needle.

In one embodiment, the administration is a subcutaneous injection with a 24 gauge needle.

In one embodiment, the administration is a subcutaneous injection with a 23 gauge needle.

In one embodiment, the plasma concentration of buprenorphine within the first hour after administration is less than 10 ng/ml.

In one embodiment, the plasma concentration of buprenorphine within the first twelve hours after administration does not exceed about 9 ng/ml.

In one embodiment, the plasma concentration of buprenorphine within the first twelve hours after administration does not exceed about 8 ng/ml.

In one embodiment, the plasma concentration of buprenorphine within the first twelve hours after administration does not exceed about 7.5 ng/ml.

In one embodiment, the plasma concentration of buprenorphine within the first twelve hours after administration does not exceed about 7 ng/ml.

In one embodiment, the plasma concentration of buprenorphine within the first twelve hours after administration does not exceed about 6.5 ng/ml.

In one embodiment, the plasma concentration of buprenorphine within the first twelve hours after administration does not exceed about 6 ng/ml.

In one embodiment, the plasma concentration of buprenorphine within the first twelve hours after administration does not exceed about 5.5 ng/ml.

In one embodiment, the plasma concentration of buprenorphine within the first twelve hours after administration does not exceed about 5 ng/ml.

In one embodiment, the plasma concentration of buprenorphine during hours 1 through 12 hours after administration does not vary by more than 10 ng/ml from the lowest to the highest concentration achieved.

In one embodiment, the plasma concentration of buprenorphine from 30 minutes to about 12 hours after administration does not vary by more than 10 ng/ml from the lowest to the highest concentration achieved.

In one embodiment, the plasma concentration of buprenorphine remains near or substantially near a therapeutic level for from between days 2 through day 30 after administration.

In one embodiment, the plasma concentration of buprenorphine remains at a substantially therapeutic level from between about day 2 through day 30 after administration.

In one embodiment, the plasma concentration of buprenorphine remains at a substantially therapeutic level from between about 12 hours through about day 35 after administration.

In one embodiment, the plasma concentration of buprenorphine remains at a substantially therapeutic level from between about day 5 through about day 40 after administration.

In one embodiment, the plasma concentration of buprenorphine remains at a substantially therapeutic level from between about day 2 through about day 30, or about 40, or about 60, or about day 120, or about day 180 after administration.

In one embodiment, the plasma concentration of buprenorphine remains at a substantially therapeutic level from between about 12 hours through about day 30, or about day 40, or about 60, or about day 120, or about day 180 after administration.

In one embodiment, the therapeutic level comprises from about 1 ng/ml to about 8 ng/ml. In one embodiment, the therapeutic level comprises from about 1 ng/ml to about 8 ng/ml for the treatment of opioid dependence. In one embodiment the therapeutic level comprises from about 1 ng/ml to about 5 ng/ml for the treatment of pain. In one embodiment, the therapeutic level comprises from about 1 ng/ml to about 8 ng/ml for the treatment of pain in opioid dependent subjects.

In one embodiment, the Cₘₐₓ does not exceed 10 ng/ml, 9 ng/ml, 8 ng/ml, 7 ng/ml, 6.25 ng/ml, 6 ng/ml, 5.75 ng/ml, 5.5 ng/ml, 5.25 ng/ml, 5 ng/ml, 4.75 ng/ml, 4 ng/ml, 3 ng/ml, or 2 ng/ml.

In one embodiment, the SRBM formulation comprises particles having diameters between about 12 to about 100 μm.

In one embodiment, the SRBM formulation comprises particles having diameters of between about 15 to about 80 μm.

In one embodiment, the SRBM formulation comprises particles, wherein the majority of the particles have diameters between about 20 and about 40 μm.

Other embodiments are disclosed infra.

### BRIEF DESCRIPTION OF THE DRAWINGS

FIG. 1 shows buprenorphine plasma concentration (ng/mL)×Time (hr) (Group 1-1068-57).

FIG. 2 shows buprenorphine plasma concentration (ng/mL)×Time (hr) (Group 2-1068-60).

FIG. 3 shows buprenorphine plasma concentration (ng/mL)×Time (hr) (Group 3-1068-78).

FIG. 4 shows buprenorphine plasma concentration (ng/mL)×Time (hr): Combined Sexes for Group 1, Group 2, and Group 3 (Linear Scale).

FIG. 5 shows buprenorphine plasma concentration (ng/mL)×Time (hr): Combined Sexes for Group 1, Group 2, and Group 3 (Log Scale).

FIG. 6 shows that after administration the buprenorphine depot injection shows stable release kinetics and stable plasma levels near a therapeutic target level for about 35 days.

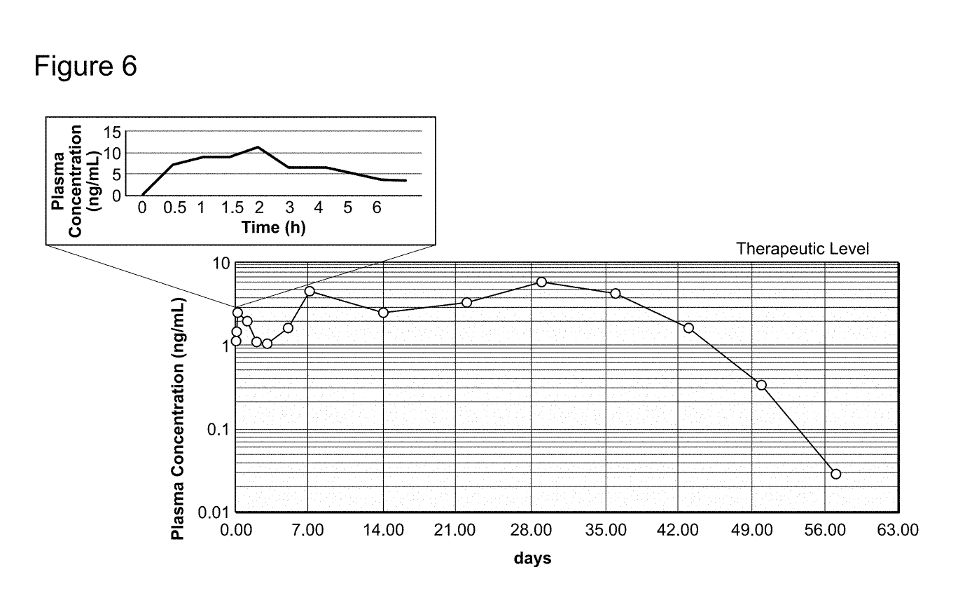

FIG. 7 shows the plasma buprenorphine concentration v. time profile following single doses SRBM formulation administration in Gottingen minipigs.

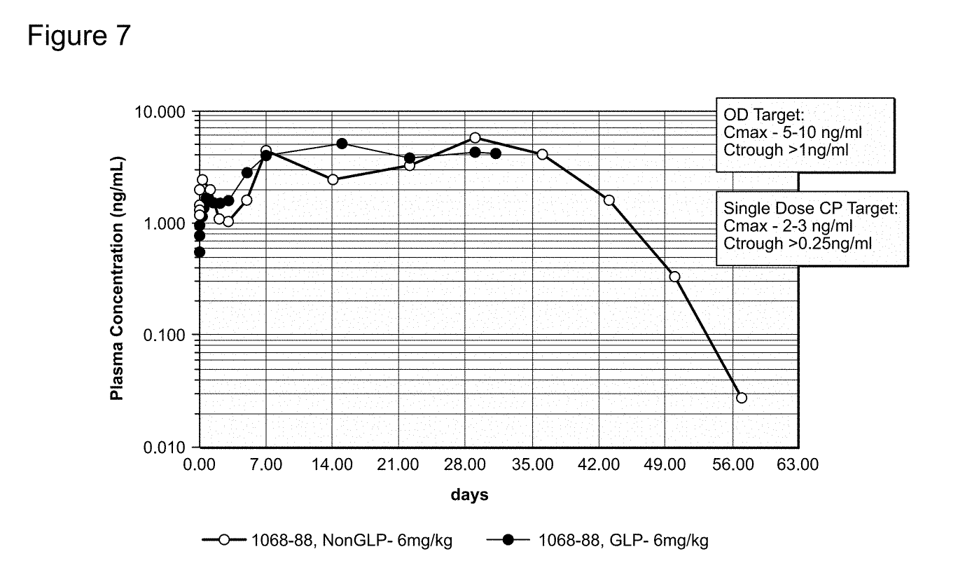

FIG. 8 shows the 24 h burst effect for the SRBM formulation upon administration of the first dose.

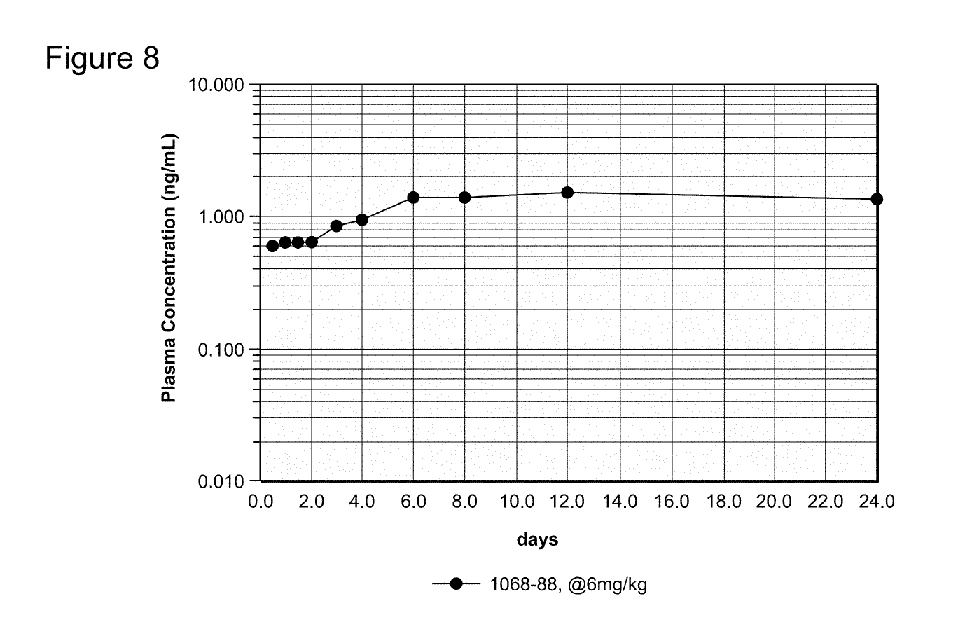

FIG. 9 shows the size distribution of the SRBM formulation.

FIG. 10 shows the SRBM formulation in a 27 gauge needle.

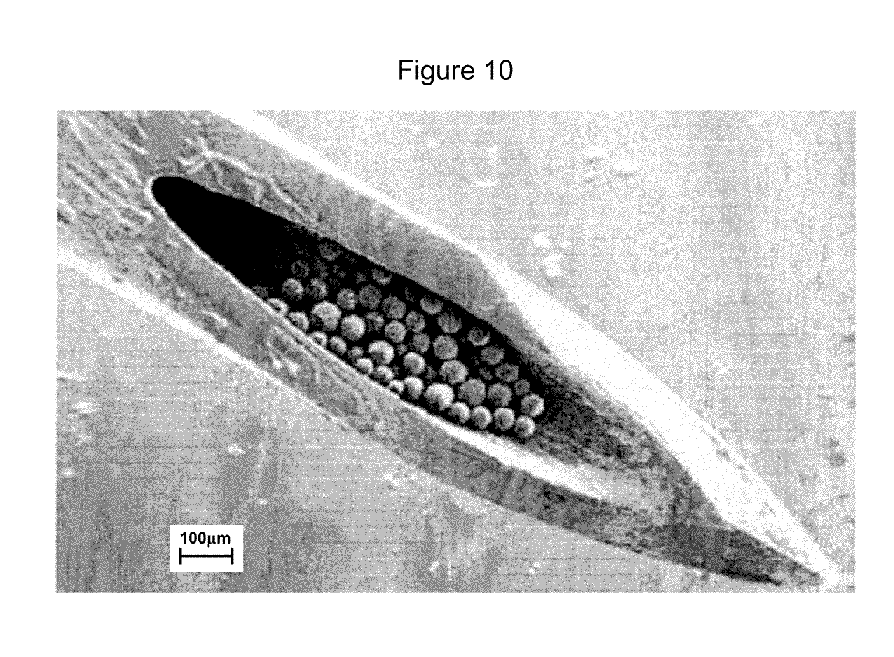

### DETAILED DESCRIPTION

Described herein are methods of treating a subject for opioid dependence and/or pain with a formulation having a sustained release profile as evidenced by the study to evaluate the pharmacokinetics (PK) of plasma buprenorphine and its major metabolite norbuprenorphine for up to 60 days following a single administration of two 3 mg/kg subcutaneous injections of Sustained Release Buprenorphine Depot Microspheres (SRBM) in male and female Gottingen minipigs.

### List of Abbreviations:

AUC_{0-INF} (hr*ng/mL) Area under the concentration-time curve from time zero up to ∞ with extrapolation of the terminal phase
AUC₀₋ₗₐₛₜ (hr*ng/mL) Area under the plasma concentration-time curve from time 0 to the time of last quantifiable plasma concentration level
AUC % extrapolated Area under the concentration-time curve extrapolated from time of last analytically quantifiable concentration to ∞ in % of the total AUC
BLQ Below the lower limit of quantitation. Refers to analyte concentrations that are below the lower limit of quantitation, that is, below the lowest calibration standard on the standard curve.
BUP Buprenorphine
Cₘₐₓ/D Cₘₐₓ normalized by dose level
Cₗₐₛₜ (ng/mL) Last analytically quantifiable plasma or serum concentration above LOQ
CL_Fobs (mL/hr/kg) Apparent total plasma or serum clearance of drug after administration
LLOQ Lower Limit Of Quantitation. The lowest calibration standard on the standard curve
MV Missing Value
N Number
NObs Number of observations
NorBUP Norbuprenorphine
SRBM Sustained Release Buprenorphine Microspheres
ULOQ Upper Limit Of Quantitation. The highest calibration standard on the standard curve

Dosage forms and formulations described herein may be made by, for example, one or more of the following methods described in:

U.S. Pat. No. 6,495,155; U.S. Pat. No. 8,916,196; U.S. Pat. No. 8,703,843; US 2010-0069602 Al; US 2010-0189800 A1; US 2013-0059008 A1; US 2012-0178629 A1; US 2011-0204533 A1; US 2015-0072928 A1 and US 2012-0082731 A1, all of which are incorporated by reference in their entirety. Microparticles may be prepared by the process as described in U.S. Pat. No. 5,407,609, which is incorporated by reference in its entirety.

In one embodiment, for a human subject the dose administered will be from about 60 mg to 240mg for a 20 to about a 40 day dose. For reference, about a 240 mg dose of SRBM will correspond to a subject taking 16-24 mg daily of an oral film or oral sublingual tablet formulation. In another embodiment, about a 180 mg dose of SRBM will correspond to a subject taking 12-16 mg of an oral film or oral sublingual tablet. In another embodiment, a 180 mg dose of SRBM will correspond to a subject taking an 8-12 mg dose of an oral film or oral sublingual tablet formulation. In another embodiment, a 60 mg dose of SRBM will correspond to a subject taking a subject taking a 4-8 mg daily dose of an oral film or oral sublingual tablet.

Excipients, release modifiers, plasticizers, pore forming agents, gelation liquids, non-active extenders, and other ingredients may also be included within the buprenorphine sustained release delivery system. Upon administration of the flowable composition, some of these additional ingredients, such as gelation liquids and release modifiers should remain with the implant, while others, such as pore forming agents should separately disperse and/or diffuse along with the organic liquid.

Exemplary formulations include, for example, buprenorphine free base, buprenorphine hydrochloride, or a metabolite or derivative thereof; microspheres made of, for example, one or more of Resomer RG 503H, Resomer RG 502H, poly(D,L-lactide-co-glycolide) having a MW 24,000-38,000 or a MW 7,000-17,000, or a poly(D,L-lactide); polyvinyl alcohol; ethyl acetate or another pharmaceutically acceptable solvent, and water. The poly(D,L-lactide-co-glycolide) used to manufacture the microspheres may be acid terminated. The poly(D,L-lactide-co-glycolide) may have a lactide:glycolide of 50:50. The poly(D,L-lactide-co-glycolide) may have a Mw 24,000-38,000 or a Mw 7,000-17,000. A mixture of poly(D,L-lactide-co-glycolide)s may be used. The ratios may be, for example, from 1:99 or 99:1 of Mw 24,000-38,000:Mw 7,000-17,000. In one embodiment, the ratio of poly(D,L-lactide-co-glycolide)s having Mw 24,000-38,000:Mw 7,000-17,000 may be 1:1, 0:1, 1:0, or any ratio there between.

The microparticles are formulated in an appropriate vehicle, exemplary vehicle formulations can be found in the Example, which follow. Buprenorphine free base, buprenorphine hydrochloride, or a metabolite or derivative thereof may be from about 10 mg to 1 g for a single dose. The formulation may further comprise naltrexone, which may range from about 150 to 300 mg. The vehicle may be sterile water, phosphate buffered saline, or other vehicle, as exemplified in the Examples, for administering the microparticles. Additives may be present to improve suspendibility of the microparticles, slow the microparticles settling, and diminish discomfort from the injection. Mannitol may be present in about 2 to 10 weight % of the vehicle. Other physiologically acceptable additives may include nonionic detergents, e.g. Tween, may be present in from about 0 to about 0.05 to 0.2 weight % of vehicle, viscosity enhancing agents, e.g. carboxymethylcellulose, in the range of about 0.1 to 1 weight % of vehicle, and other additives, as appropriate. The amount of vehicle will generally be in the range of about 1 to 5 mL, usually 1 to 3.5 mL. The microparticles are dispersed in the vehicle immediately before use. The sterile microparticles may be stored in a sterile vial with a septum, where the microparticles may be mixed with the vehicle and then withdrawn into a syringe.

The microparticles may be prepared by a process that is an emulsion-based process, which involves the preparation of an emulsion comprising an aqueous continuous phase (water and a surfactant and/or thickening agent) and a hydrophobic phase (polymer solvent, polymer and drug). After formation of the emulsion, the polymer solvent is extracted into an aqueous extraction phase. After a sufficient amount of polymer solvent is extracted to harden the microparticles, the microparticles are collected on sieves and washed to remove any surfactant remaining on the surface of the microparticles. The microparticles are then dried with a nitrogen stream for an extended period, e.g. about 12 hours, then dried in a vacuum oven at room temperature until at least substantially dry, conveniently for about 3 days.

Using storage containers for the different streams, tubing, three-way valves and a homogenizer, the microparticle manufacturing system is assembled. The organic solution is introduced into a first tube connected to a three way valve, which connects to the aqueous continuous phase and to the homogenizer. By controlling the rate of flow of the two streams into the line connecting the homogenizer, the ratio of the two streams can be controlled, as well as the residence time in the homogenizer. The effluent from the homogenizer exits through a line which connects to a three-way valve through which the water stream is introduced. The rate of flow ratio controls the amount of water to the homogenizer effluent stream. The residence time of the water extraction step is controlled by the length of tubing and the rate of flow of the combined streams. The microparticles are then segregated by size by passing through two or more sieves which eliminates microparticles outside the desired range.

For the preparation of the microparticles, the dispersed phase may contain, for example, about 1-10 weight % of the drug and about 20-weight % polymer dispersed or dissolved (hereinafter both are included when referring to the polymer in a solvent as dispersed) in a solvent, such as, for example, ethyl acetate. The continuous phase is an aqueous solution of about 1-10 weight % of poly(vinyl alcohol) and contains ethyl acetate at 1 to 7.5 weight %. The extraction phase may be water or another solvent. The amount of drug may be from about 10 to 50 weight % in excess of the final drug in the microparticles. Temperatures may be ambient, for example, from about 15 to 30° C.

After the microparticles have been collected and dried they may be stored at ambient temperatures, particularly in the range of about 0-20° C. in an oxygen and water free environment, or divided into aliquots into appropriate containers and sterilized. Various methods of sterilization may be employed, gamma irradiation being convenient.

The SRBM formulations may be administered by an intramuscular injection or a subcutaneous injection. The pharmaceutically effective amount of the formulation is directly injected into a pharmaceutically acceptable site, e.g. gluteus. Thereafter, the subject may be monitored for drug plasma concentration to ensure that the amount is in the therapeutic range. When the drug plasma concentration falls below the therapeutic range, a subsequent injection may be made and this process repeated during the treatment period. The therapeutic period may be from 1-5 days, 15 days, 30 days, 45 days, 60 days, 90 days, 120 days, 180 days, from 1-30 days, 1-60 days, 1-90 days, 1-120 days, or from 1-180 days.

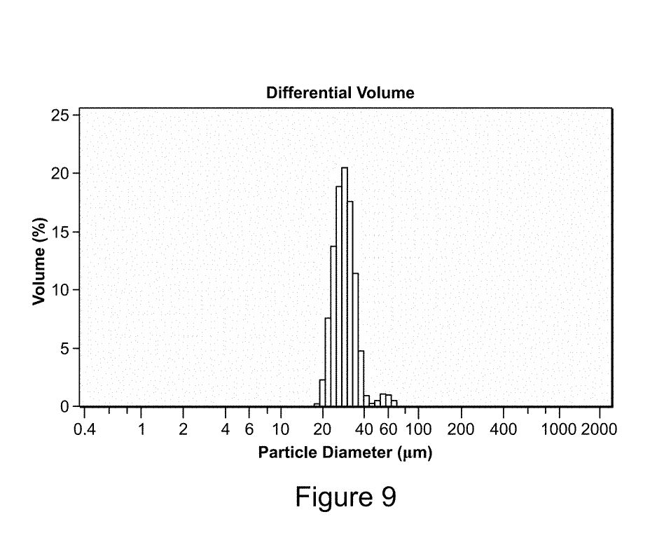

The formulations of SRBM disclosed herein are syrangable, for example, the particle diameter and particle distribution are shown in FIG. 9. This enables the administration of the SRBM formulation in a needle that is from between a 23 and 27 gauge. In one embodiment, the formulation is administered in a 27 gauge needle. In another embodiment the formulation is administered in a 26 gauge needle. In another embodiment the formulation is administered in a 25 gauge needle. In another embodiment the formulation is administered in a 24 gauge needle. In another embodiment, the formulation is administered in a 23 gauge needle.

In one embodiment, the formulation is for about 30 days of treatment of opioid dependence. For example, the formulation will have a Cₘₐₓ of between about 5 to about 10 ng/ml and C_{trough} of about >1 ng/ml. In one embodiment, the formulation is for about 30 days for treatment of pain in an opioid dependent subject. For example, the formulation will have a Cₘₐₓ of about 2 to about 3 ng/ml and a C_{trough} of about >0.25ng/ml.

In one embodiment, the formulation is for about 30 days of treatment of chronic pain in a subject. For example, the formulation will have a Cₘₐₓ of about 2 to about 3 ng/ml and a C_{trough} of about >0.25 ng/ml. Each of these formulations, for example, for treatment of opioid dependence, pain in an opioid dependent subject and/or for chronic pain have well controlled 24-h release and also have minimal burst following administration.

In one embodiment, the treatment of opioid dependence is for maintenance treatment of opioid dependence. In another embodiment, the treatment of opioid dependence is for initiation of treatment of opioid dependence.

The formulation may be packaged for example in a prefilled syringe or in a vial. The vial may be dried to be reconstituted or it may be in a liquid formulation. The formulation may be administered, for example, at a physician's office, by a medical professional or by a subject or another person.

The formulation has a small injection volume, for example to reduced reaction or irritation at the injection site. Volumes are from about 0.25 ml to about 2.5 ml. For example, the volume of injection may be about ≦1.5mL. The formulation will be stored, for example, at room temperature or refrigerated or at some temperature in between.

In one embodiment, the second administration will have a burst that is additive to the subject's plasma concentration from a previous administration.

In one embodiment, the burst is less than 10 mg/ml above the subject's plasma concentration at the time of administration of the second dose.

In one embodiment, the burst is from between about 4 ng/ml to about 10 mg/ml above the subject's plasma concentration at the time of administration of the second dose.

As used herein, burst includes an initial absorption of an active pharmaceutical ingredient from a sustained release product that is higher than the dose that will be released at steady state. Burst, also includes, an initial rapid release of drug that then plateaus to a therapeutic level or near a therapeutic level. The formulations described herein minimize the burst effect. The formulations described herein avoid toxic levels being released upon administration. The formulations also avoid side-effects of the burst effects, such as nausea and/or vomiting. The burst of the first administration (assuming no buprenorphine taken prior to the first dose or all previously administered buprenorphine is no longer present or detectable) may be determined, for example by measuring blood levels. On second and subsequent doses or dose exposures, there will be an existing concentration at the end of the prior dose interval. The burst would be, for example, the amount that the plasma concentration increases from this point.

The SRBM formulations described herein comprise particle having diameters of between about 15 to about 80 μm. In one embodiment, the particles have diameters between about 12 to about 100 μm. In one embodiment, the majority of particles have diameters between about 20 and about 40 μm.

### EXAMPLES

An objective of this portion of the toxicology study was to evaluate the pharmacokinetics (PK) of plasma buprenorphine and its major metabolite norbuprenorphine for up to 60 days following a single administration of two 3 mg/kg subcutaneous injections of Sustained Release Buprenorphine Depot Microspheres (SRBM) on Day 1 in male and female Gottingen minipigs. The PK of three (3) different formulations of SRBM were evaluated and compared.

Blood samples were collected at time 0 (prior to dosing) and at 0.5, 1, 1.5, 2, 3, 4, 6, 8, 12 and 24 hours following dosing. Blood samples were also collected at 48, 72, 120 and 168 hours post-dose and approximately weekly thereafter for the remainder of the study. Plasma concentration×time data buprenorphine was evaluated.

Cₘₐₓ values were markedly lower for Group 1 formulation 1068-57 (5.3-7.1 ng/mL) compared to Group 2 formulation 1068-60 (25.0-32.5 ng/mL) and Group 3 formulation 1068-78 (8.8-32.5 ng/mL). Tₘₐₓ values were greater for Group 1 formulation 1067-58 (168-696 hr) compared to Group 2 formulation 1067-60 and Group 3 formulation 1067-78 (120-168 hr). Taking into account the amount of variability in each of the groups, the total exposure to buprenorphine (AUC_{0-INF}) was similar when Groups 1, 2 and 3 were compared. Although total exposure to buprenorphine was similar when the 3 groups were compared, the shape of the plasma concentration×time curves were much different when comparing Group 1 to Groups 2 and 3. Cₘₐₓ values were much lower and Tₘₐₓ values were longer for Group 1 compared to Groups 2 and 3. The terminal half-life of elimination for buprenorphine was lowest and least variable for Group 1 formulation 1068-57 (57-79 hr) compared to Group 2 formulation 1068-60 (59-270 hr) and Group 3 formulation 1068-78 (78-186 hr). Exposure to quantifiable plasma norbuprenorphine concentrations for all 3 groups was low (from below the lower limit of quantitation (LLOQ) to approximately 7 fold greater than the LLOQ of 0.05 ng/mL), however the plasma concentrations were least for Group 1 formulation 1068-57 when compared to Group 2 formulation 1068-60 and Group 3 formulation 1068-78.

The study consisted of three treated groups, with 2 animals/sex/group. The animals received two 3 mg/kg subcutaneous injections (total of 6 mg/kg) on Day 1 followed by at least 60 days of observation and scheduled blood collections to evaluate the PK of buprenorphine and its major metabolite norbuprenorphine. The study design is summarized in the table below:

TABLE 1
Study Design

| | | | | Dose | |
|---|---|---|---|---|---|
| | | Total | SRBM | Volume/ | Number |
| | | SRBM | Buprenorphine | Injection | of |
| Group | Group | Dose | Concentration | Site (2 | Animals/ |
| Number | Name | (mg/kg) | (mg/mL) | Sites) | Group |
| 1 | Buprenorphine | 6 | 30 | 0.1 mL/site | 2/sex |
| | Depot | | | | |
| | 1068-57 | | | | |
| 2 | Buprenorphine | 6 | 30 | 0.1 mL/site | 2/sex |
| | Depot | | | | |
| | 1068-60 | | | | |
| 3 | Buprenorphine | 6 | 30 | 0.1 mL/site | 2/sex |
| | Depot | | | | |
| | 1068-78 | | | | |

Vehicle: The vehicle was a liquid and was used as supplied. The vehicle contained Na-CMC (5 mg), D-Mannitol (50 mg), Tween 80 (1 mg), and WFI (Water for Injection; 1 g).
Test Material: The test material was Sustained Release Microspheres (SRBM) and was supplied as three formulations.

The test materials were re-suspended in vehicle (on the day of dosing) to achieve concentrations of each of the test materials of 30 mg/mL. Animals received two (2) subcutaneous 3 mg/kg injections of one of the following SRBM formulations: 30 mg/mL 1068-57, 30 mg/mL 1068-60, and 30 mg/mL 1068-78, equivalent to a total dose of 6 mg/kg SRBM when administered at a dose volume of 0.1 mL/kg/injection site (2 injection sites).

TABLE 2
SRBM Formulations

| | 1068-57 | 1068-60 | 1068-78 | 1068-90 | |
|---|---|---|---|---|---|
| Component | (grams) | (grams) | (grams) | (grams) | Function |
| Buprenorphine | 12.5 | 5 | 5 | 12.5 | Active drug |
| free base EP | | | | | |
| Resomer RG | 12.5 | 2.5 | 3.6 | 0 | Sustained |
| 503H | | | | | release polymer |
| Resomer RG | 0 | 2.5 | 1.43 | 12.5 | Sustained |
| 502H | | | | | release polymer |
| Polyvinyl | 5 | 2.5 | 2.5 | 5 | Surfactant for |
| alcohol NF | | | | | emulsion |
| Ethyl acetate NF | 348.3 | 147.6 | 147.6 | 249.1 | Solvent for |
| for DP & CP | | | | | polymers, and |
| | | | | | saturate CP |
| Water for | 45 L | 18 L | 18 L | 28 L | Extraction phase |
| Injection | | | | | |

TABLE 3
Components and Composition of
Vehicle Used for Reconstitution

| Component | Amount | | Composition (%) |
|---|---|---|---|
| Sodium | 5 | mg | 0.5 |
| carboxymethylcellulose NF | | | |
| D-Mannitol NF | 50 | mg | 5 |
| Tween 80 | 1 | mg | 0.1 |
| Water for Injection | 1 | g | |

| | | |
|---|---|---|
| Glacial acetate acid | As needed | NA |

### Pharmacokinetics (PK)

Whole blood was collected at each sample time point. The blood samples were prepared for analysis. Phoenix WinNonlin®, version 6.3 (Pharsight Corporation, Mountain View, Calif.) was used to evaluate plasma buprenorphine PK parameters. There were insufficient levels of plasma norbuprenorphine to conduct a PK evaluation of this metabolite. Nominal dose levels and blood collection times were employed for the PK evaluation of buprenorphine. Plasma buprenorphine concentrations below the lower limit of quantitation (0.05 ng/mL) were assigned a value of 0.

A comparison of buprenorphine pK parameters after single subcutaneous injection of 6 mg/kg of formulations 1068-57 (Group 1), 1068-60 (Group 2), and 1068-78 (Group 3)

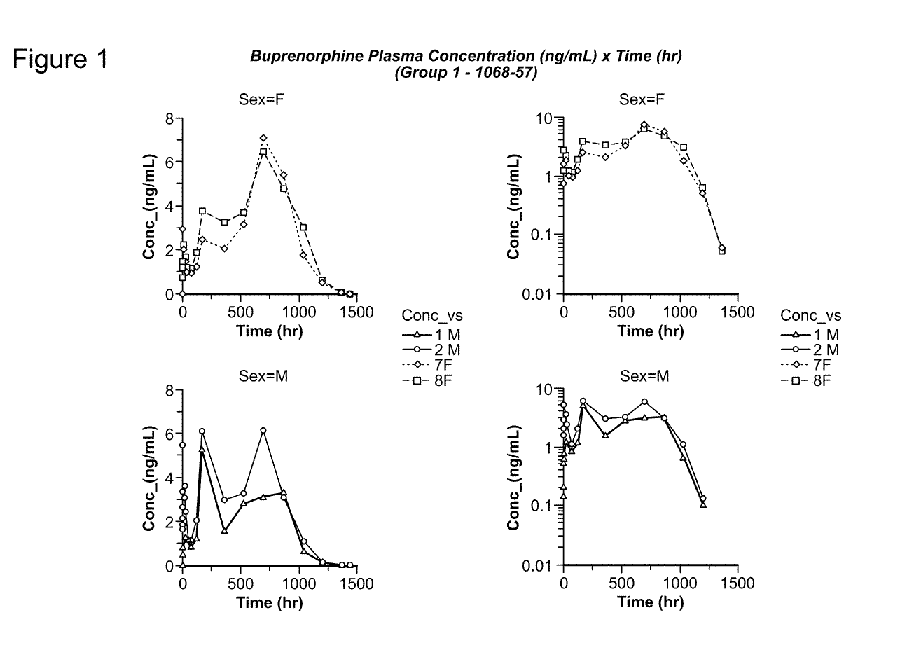

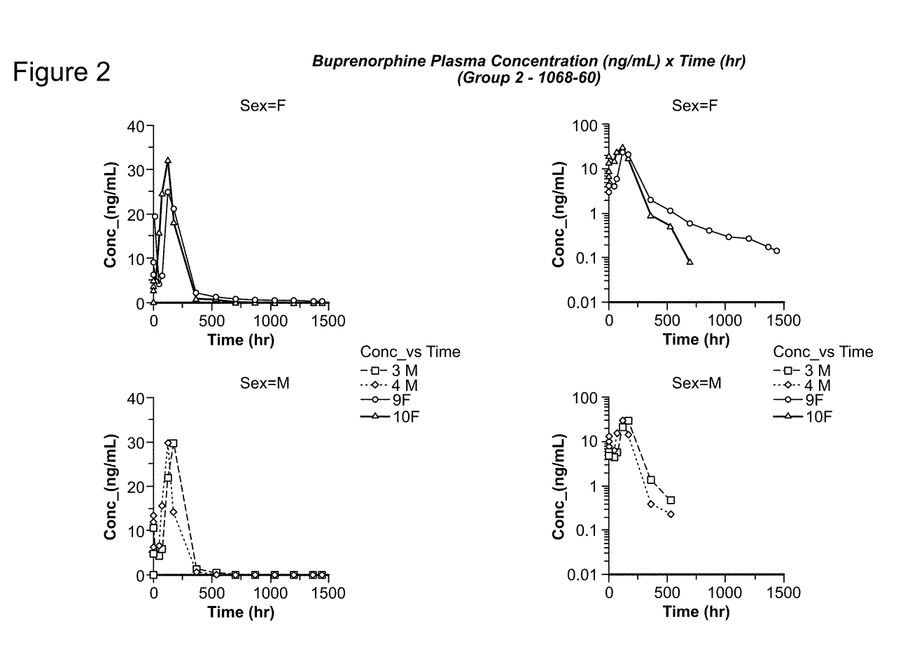

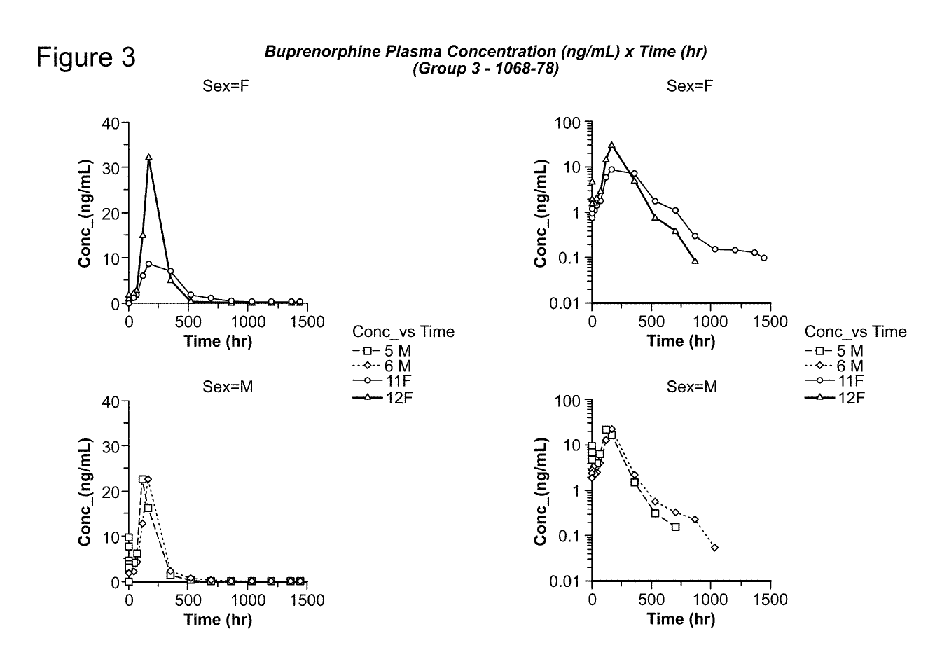

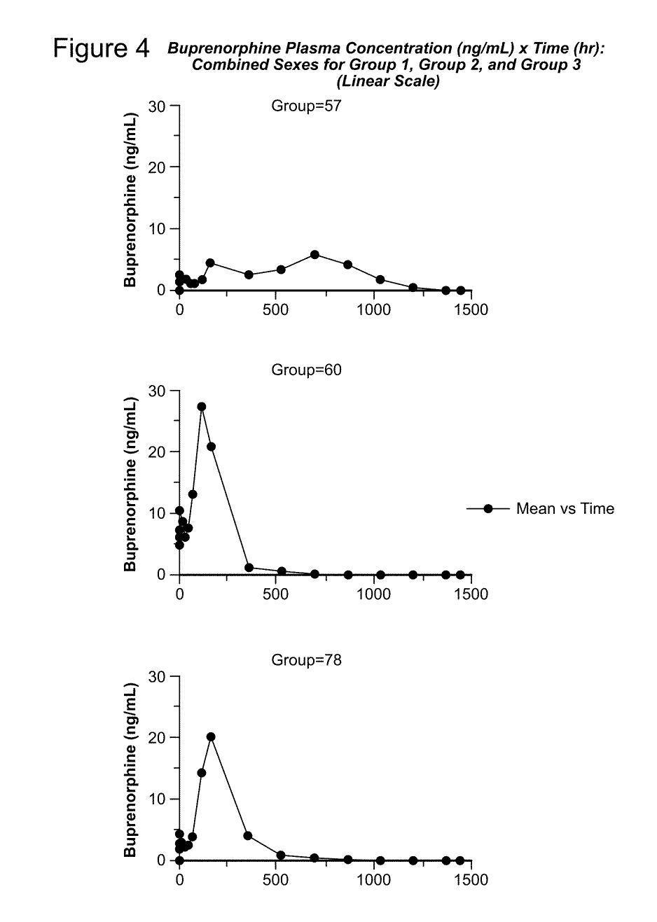

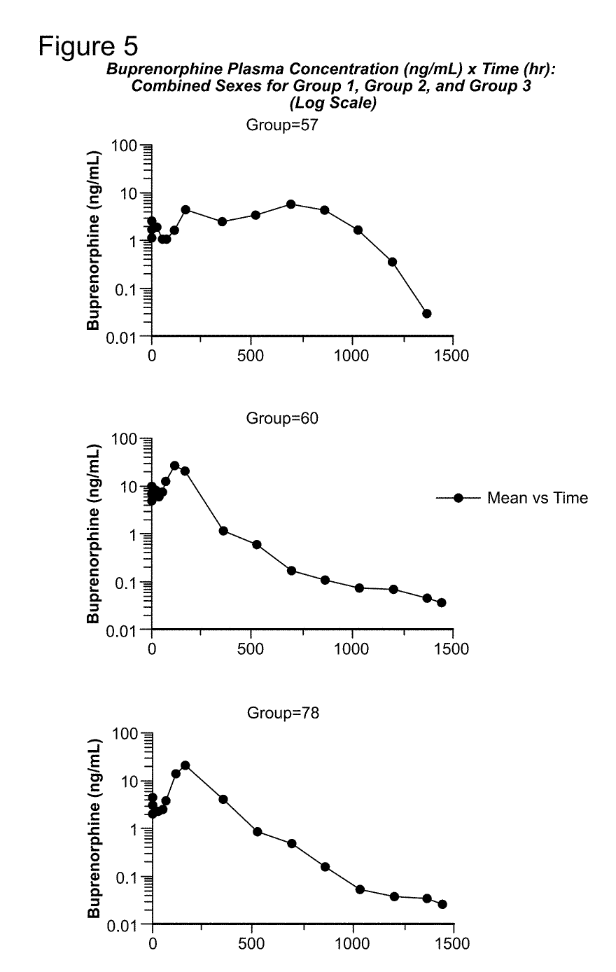

(Tables 4-6 and FIGS. 1-5) were conducted. Cₘₐₓ values were markedly lower for Group 1 formulation 1068-57 (5.3-7.1 ng/mL) compared to Group 2 formulation 1068-60 (25.0-32.5 ng/mL) and Group 3 formulation 1068-78 (8.8-32.5 ng/mL). There were no apparent gender-related differences in Cₘₐₓ values in Groups 1 and 2.

Tₘₐₓ values were greater for Group 1 formulation 1067-58 (168-696 hr) compared to Group 2 formulation 1067-60 and Group 3 formulation 1067-78 (120-168 hr). There were no apparent gender-related differences in Tₘₐₓ values for Groups 2 and 3.

Tₗₐₛₜ values were variable for Group 2 formulation 1068-60 (528-1440 hr). Group 3 formulation 1068-78 Tₗₐₛₜ values were also variable, ranging from 696-1440 hr. Group 1 formulation 1068-57 Tₗₐₛₜ values were least variable, ranging from 1200-1368 hr.

Taking into account the amount of variability in each of the groups, the total exposure to buprenorphine (AUC_{0-INF}) was similar when Groups 1, 2 and 3 were compared. The values ranged from 2613-4300 hr*ng/mL for Group 1 formulation 1068-57, 3457-4789 hr*ng/mL for Group 2 formulation 1068-60, and 3281-5104 hr*ng/mL for Group 3 formulation 1068-78. Although total exposure to buprenorphine appears similar when the 3 groups are compared, the shapes of the plasma concentration x time curves are different when comparing Group 1 to Groups 2 and 3. Cₘₐₓ values were much lower and Tₘₐₓ values were longer for Group 1 compared to Groups 2 and 3.

Taking into account the variability in each of the groups, clearance values (Cl_Fobs) appeared similar for Groups 1-3. Clearance remains constant with first-order elimination kinetics. The amount of drug eliminated per unit time changes with the concentration of drug in the plasma.

The terminal half-life of elimination for buprenorphine was shortest and least variable for Group 1 formulation 1068-57 (57-79 hr) compared to Group 2 formulation 1068-60 (59-270 hr) and Group 3 formulation 1068-78 (78-186 hr). The variability in Group 2 was primarily due to female number 9 (t_{1/2}, 270 hr) compared to the other animals in the group (59-72 hr).

A comparison of norbuprenorphine plasma concentrations after single subcutaneous injection of 6 mg/kg SRBM formulations 1068-57 (Group 1), 1068-60 (Group 2), and 1068-78 (Group 3) (Tables 7-9) was conducted. Low plasma concentrations of norbuprenorphine (0.05-0.07 ng/mL) were observed for Group 1 formulation 1068-57 and coincided with maximal plasma buprenorphine concentrations (Day 7 for male animals 1 and 2; Day 29 for male animal 2 and female animals 7 and 8). There were no other measurable plasma concentrations of norbuprenorphine above the LLOQ (0.05 ng/mL) observed at any other time point throughout the study for Group 1 animals.

Quantifiable plasma norbuprenorphine concentrations were observed for Group 2 formulation 1068-60 beginning as early as 0.5 hr post-dose and lasting as long as Day 7 (male animals 3 and 4, and female animal 10) or Day 15 (female animal 9). The male plasma norbuprenorphine concentrations ranged from 0-0.32 ng/mL while the female values ranged from 0-0.36 ng/mL. The duration of exposure (number of days) and level of exposure (ng/mL plasma norbuprenorphine) were much greater for Group 2 compared to Group 1.

Low concentrations of plasma norbuprenorphine were observed as early as 0.5 hr post-dose for Group 3 formulation 1068-78 for female number 5 (0.10 ng/mL) and male number 12 (0.16 ng/mL). All four animals had quantifiable plasma norbuprenorphine concentrations on Days 5 and 7 (0.10-0.17 ng/mL for the males and 0.07-0.20 ng/mL for the females). Female number 11 continued to have quantifiable plasma norbuprenorphine levels through Day 60 (0.07-0.26 ng/mL), which was the final sample time point for the study. The duration of exposure (number of days) and level of exposure (ng/mL plasma norbuprenorphine) were greater for Group 3 compared to Group 1, but were less than for Group 2.

### Conclusions

Cₘₐₓ values were markedly lower for Group 1 formulation 1068-57 (5.3-7.1 ng/mL) compared to Group 2 formulation 1068-60 (25.0-32.5 ng/mL) and Group 3 formulation 1068-78 (8.8-32.5 ng/mL). Tₘₐₓ values were greater for Group 1 formulation 1067-57 (168-696 hr) compared to Group 2 formulation 1067-60 and Group 3 formulation 1067-78 (120-168 hr). Taking into account the amount of variability in each of the groups, the total exposure to buprenorphine (AUC_{0-INF}) was similar when Groups 1, 2 and 3 were compared.

Although total exposure to buprenorphine was similar when the 3 groups were compared, the shape of the plasma concentration x time curves were much different when comparing Group 1 to Groups 2 and 3. Cₘₐₓ values were much lower and Tₘₐₓ values were longer for Group 1 compared to Groups 2 and 3.

The terminal half-life of elimination for buprenorphine was lowest and least variable for Group 1 formulation 1068-57 (57-79 hr) compared to Group 2 formulation 1068-60 (59-270 hr) and Group 3 formulation 1068-78 (78-186 hr).

Exposure to quantifiable plasma norbuprenorphine concentrations for all 3 groups was low (from below the LLOQ of 0.05 ng/mL to approximately 7 fold greater than the LLOQ), however it was least for Group 1 formulation 1068-57 compared to Group 2 formulation 1068-60 and Group 3 formulation 1068-78.

TABLE 4
Plasma Buprenorphine Concentration (ng/mL) × Time (hr): Group 1
(6 mg/kg 1068-57)

| | | #1 Male | #2 Male | #7 Female | #8 Female |
|---|---|---|---|---|---|
| Time | Time | Plasma BUP | Plasma BUP | Plasma BUP | Plasma BUP |
| (day) | (hr) | (ng/mL) | (ng/mL) | (ng/mL) | (ng/mL) |
| 1 | 0 | 0.00 | 0.00 | 0.00 | 0.00 |
| 1 | 0.5 | 0.56 | 1.88 | 1.47 | 0.76 |
| 1 | 1 | 0.15 | 1.66 | 1.39 | 2.10 |
| 1 | 1.5 | 0.15 | 2.13 | 1.05 | 1.13 |
| 1 | 2 | 0.22 | 2.65 | 1.61 | 1.31 |
| 1 | 3 | 0.57 | 3.38 | 2.93 | 1.88 |
| 1 | 4 | 0.78 | 5.46 | 1.63 | 1.88 |
| 1 | 6 | 0.87 | 2.68 | 1.92 | 2.40 |
| 1 | 8 | 0.67 | 3.05 | 2.00 | 2.09 |
| 1 | 12 | 0.84 | 3.59 | 1.75 | 1.30 |
| 1 | 24 | 1.27 | 2.46 | 1.94 | 2.24 |
| 2 | 48 | 1.14 | 1.10 | 0.97 | 1.18 |
| 3 | 72 | 0.90 | 1.16 | 0.98 | 1.14 |
| 5 | 120 | 1.25 | 2.05 | 1.23 | 1.89 |
| 7 | 168 | 5.30 | 6.10 | 2.46 | 3.77 |
| 15 | 360 | 1.56 | 2.98 | 2.05 | 3.27 |
| 22 | 528 | 2.86 | 3.25 | 3.15 | 3.70 |
| 29 | 696 | 3.13 | 6.12 | 7.10 | 6.47 |
| 36 | 864 | 3.30 | 3.08 | 5.39 | 4.77 |
| 43 | 1032 | 0.67 | 1.09 | 1.78 | 3.00 |
| 50 | 1200 | 0.11 | 0.13 | 0.50 | 0.60 |
| 57 | 1368 | 0.00 | 0.00 | 0.06 | 0.05 |
| 60 | 1440 | 0.00 | 0.00 | 0.00 | 0.00 |

TABLE 5
Plasma Buprenorphine Concentration (ng/mL) × Time (hr): Group 2
(6 mg/kg 1068-60)

| | | #3 Male | #4 Male | #6 Female | #10 Female |
|---|---|---|---|---|---|
| Time | Time | Plasma BUP | Plasma BUP | Plasma BUP | Plasma BUP |
| (day) | (hr) | (ng/mL) | (ng/mL) | (ng/mL) | (ng/mL) |
| 1 | 0 | 0.00 | 0.00 | 0.00 | 0.00 |
| 1 | 0.5 | 4.84 | 10.90 | 8.13 | 5.55 |
| 1 | 1 | 6.46 | 6.43 | 3.00 | 3.70 |
| 1 | 1.5 | 5.78 | 10.00 | 3.44 | 4.79 |
| 1 | 2 | 6.62 | 5.93 | 9.14 | 4.56 |
| 1 | 3 | 13.30 | 6.49 | 6.33 | 15.30 |
| 1 | 4 | 10.80 | 13.20 | 4.53 | 4.97 |
| 1 | 6 | 6.01 | 6.40 | 3.44 | 4.19 |
| 1 | 8 | 12.70 | 4.83 | 3.53 | 4.86 |
| 1 | 12 | 5.52 | 5.54 | 19.40 | 4.87 |
| 1 | 24 | 5.07 | 4.84 | 5.17 | 9.91 |
| 2 | 48 | 4.34 | 6.38 | 4.16 | 15.90 |
| 3 | 72 | 6.02 | 15.70 | 6.01 | 24.70 |
| 5 | 120 | 21.90 | 29.90 | 25.00 | 32.50 |
| 7 | 168 | 29.80 | 14.20 | 21.10 | 18.10 |
| 15 | 360 | 1.35 | 0.39 | 2.03 | 0.94 |
| 22 | 528 | 0.46 | 0.23 | 1.14 | 0.54 |
| 29 | 696 | 0.00 | 0.00 | 0.59 | 0.08 |
| 36 | 864 | 0.00 | 0.00 | 0.42 | 0.00 |
| 43 | 1032 | 0.00 | 0.00 | 0.29 | 0.00 |
| 50 | 1200 | 0.00 | 0.00 | 0.27 | 0.00 |
| 57 | 1368 | 0.00 | 0.00 | 0.18 | 0.00 |
| 60 | 1440 | 0.00 | 0.00 | 0.15 | 0.00 |

TABLE 6
Plasma Buprenorphine Concentration (ng/mL) × Time (hr): Group 3
(6 mg/kg 1068-78)

| | | #5 Male | #6 Male | #11 Female | #12 Female |
|---|---|---|---|---|---|
| Time | Time | Plasma BUP | Plasma BUP | Plasma BUP | Plasma BUP |
| (day) | (hr) | (ng/mL) | (ng/mL) | (ng/mL) | (ng/mL) |
| 1 | 0 | 0.00 | 0.00 | 0.00 | 0.00 |
| 1 | 0.5 | 9.79 | 1.99 | 0.75 | 4.93 |
| 1 | 1 | 7.93 | 1.95 | 1.12 | 1.30 |
| 1 | 1.5 | 3.77 | 1.96 | 0.91 | 1.22 |
| 1 | 2 | 4.70 | 1.86 | 0.89 | 1.30 |
| 1 | 3 | 3.70 | 2.16 | 0.94 | 1.44 |
| 1 | 4 | 3.65 | 2.43 | 1.14 | 1.71 |
| 1 | 6 | 3.39 | 2.72 | 1.28 | 1.81 |
| 1 | 8 | 2.83 | 4.88 | 1.71 | 1.63 |
| 1 | 12 | 2.85 | 2.21 | 1.96 | 1.48 |
| 1 | 24 | 2.85 | 2.33 | 1.96 | 1.88 |
| 2 | 48 | 4.13 | 2.12 | 1.48 | 2.31 |
| 3 | 72 | 6.34 | 4.33 | 1.81 | 2.91 |
| 5 | 120 | 22.60 | 13.00 | 6.10 | 15.40 |
| 7 | 168 | 16.40 | 22.60 | 8.78 | 32.50 |
| 15 | 360 | 1.52 | 2.15 | 7.10 | 5.15 |
| 22 | 528 | 0.31 | 0.57 | 1.76 | 0.75 |
| 29 | 696 | 0.16 | 0.32 | 1.07 | 0.40 |
| 36 | 864 | 0.00 | 0.24 | 0.29 | 0.09 |
| 43 | 1032 | 0.00 | 0.06 | 0.15 | 0.00 |
| 50 | 1200 | 0.00 | 0.00 | 0.14 | 0.00 |
| 57 | 1368 | 0.00 | 0.00 | 0.13 | 0.00 |
| 60 | 1440 | 0.00 | 0.00 | 0.10 | 0.00 |

TABLE 7
Plasma Norbuprenorphine Concentration (ng/mL) × Time (hr):
Group 1 (6 mg/kg 1068-57)

| | | | #2 Male | #7 Female | #8 Female |
|---|---|---|---|---|---|
| | | #1 Male | Plasma | Plasma | Plasma |
| Time | Time | Plasma NorBUP | NorBUP | NorBUP | NorBUP |
| (day) | (hr) | (ng/mL) | (ng/mL) | (ng/mL) | (ng/mL) |
| 1 | 0 | 0.00 | 0.00 | 0.00 | 0.00 |
| 1 | 0.5 | 0.00 | 0.00 | 0.00 | 0.00 |
| 1 | 1 | 0.00 | 0.00 | 0.00 | 0.00 |
| 1 | 1.5 | 0.00 | 0.00 | 0.00 | 0.00 |
| 1 | 2 | 0.00 | 0.00 | 0.00 | 0.00 |
| 1 | 3 | 0.00 | 0.00 | 0.00 | 0.00 |
| 1 | 4 | 0.00 | 0.00 | 0.00 | 0.00 |
| 1 | 6 | 0.00 | 0.00 | 0.00 | 0.00 |
| 1 | 8 | 0.00 | 0.00 | 0.00 | 0.00 |
| 1 | 12 | 0.00 | 0.00 | 0.00 | 0.00 |
| 1 | 24 | 0.00 | 0.00 | 0.00 | 0.00 |
| 2 | 48 | 0.00 | 0.00 | 0.00 | 0.00 |
| 3 | 72 | 0.00 | 0.00 | 0.00 | 0.00 |
| 5 | 120 | 0.00 | 0.00 | 0.00 | 0.00 |
| 7 | 168 | 0.00 | 0.06 | 0.00 | 0.00 |
| 15 | 360 | 0.00 | 0.00 | 0.00 | 0.00 |
| 22 | 528 | 0.00 | 0.00 | 0.00 | 0.00 |
| 29 | 696 | 0.00 | 0.06 | 0.06 | 0.07 |
| 36 | 864 | 0.00 | 0.00 | 0.05 | 0.00 |
| 43 | 1032 | 0.00 | 0.00 | 0.00 | 0.00 |
| 50 | 1200 | 0.00 | 0.00 | 0.00 | 0.00 |
| 57 | 1368 | 0.00 | 0.00 | 0.00 | 0.00 |
| 60 | 1440 | 0.00 | 0.00 | 0.00 | 0.00 |

TABLE 8
Plasma Norbuprenorphine Concentration (ng/mL) × Time (hr):
Group 2 (6 mg/kg 1068-60)

| | | | #4 Male | #9 Female | #10 Female |
|---|---|---|---|---|---|
| | | #3 Male | Plasma | Plasma | Plasma |
| Time | Time | Plasma NorBUP | NorBUP | NorBUP | NorBUP |
| (day) | (hr) | (ng/mL) | (ng/mL) | (ng/mL) | (ng/mL) |
| 1 | 0 | 0.00 | 0.00 | 0.00 | 0.00 |
| 1 | 0.5 | 0.12 | 0.15 | 0.27 | 0.17 |
| 1 | 1 | 0.13 | 0.10 | 0.12 | 0.10 |
| 1 | 1.5 | 0.10 | 0.15 | 0.11 | 0.11 |
| 1 | 2 | 0.10 | 0.11 | 0.16 | 0.07 |
| 1 | 3 | 0.18 | 0.11 | 0.14 | 0.18 |
| 1 | 4 | 0.12 | 0.18 | 0.08 | 0.12 |
| 1 | 6 | 0.08 | 0.09 | 0.07 | 0.09 |
| 1 | 8 | 0.15 | 0.06 | 0.07 | 0.06 |
| 1 | 12 | 0.08 | 0.07 | 0.22 | 0.07 |
| 1 | 24 | 0.06 | 0.00 | 0.06 | 0.11 |
| 2 | 48 | 0.07 | 0.08 | 0.00 | 0.17 |
| 3 | 72 | 0.08 | 0.17 | 0.10 | 0.29 |
| 5 | 120 | 0.25 | 0.28 | 0.26 | 0.34 |
| 7 | 168 | 0.32 | 0.15 | 0.36 | 0.24 |
| 15 | 360 | 0.00 | 0.00 | 0.06 | 0.00 |
| 22 | 528 | 0.00 | 0.00 | 0.00 | 0.00 |
| 29 | 696 | 0.00 | 0.00 | 0.00 | 0.00 |
| 36 | 864 | 0.00 | 0.00 | 0.00 | 0.00 |
| 43 | 1032 | 0.00 | 0.00 | 0.00 | 0.00 |
| 50 | 1200 | 0.00 | 0.00 | 0.00 | 0.00 |
| 57 | 1368 | 0.00 | 0.00 | 0.00 | 0.00 |
| 60 | 1440 | 0.00 | 0.00 | 0.00 | 0.00 |

TABLE 9
Plasma Norbuprenorphine Concentration (ng/mL) × Time (hr):
Group 3 (6 mg/kg 1068-78)

| | | #5 Male | #6 Male | #11 Female | #12 Female |
|---|---|---|---|---|---|
| | | Plasma | Plasma | Plasma | Plasma |
| Time | Time | NorBUP | NorBUP | NorBUP | NorBUP |
| (day) | (hr) | (ng/mL) | (ng/mL) | (ng/mL) | (ng/mL) |
| 1 | 0 | 0.00 | 0.00 | 0.00 | 0.00 |
| 1 | 0.5 | 0.10 | 0.00 | 0.00 | 0.16 |
| 1 | 1 | 0.07 | 0.00 | 0.00 | 0.00 |
| 1 | 1.5 | 0.07 | 0.00 | 0.00 | 0.00 |
| 1 | 2 | 0.07 | 0.00 | 0.00 | 0.00 |
| 1 | 3 | 0.00 | 0.00 | 0.00 | 0.00 |
| 1 | 4 | 0.00 | 0.00 | 0.00 | 0.00 |
| 1 | 6 | 0.00 | 0.00 | 0.00 | 0.00 |
| 1 | 8 | 0.00 | 0.00 | 0.00 | 0.00 |
| 1 | 12 | 0.00 | 0.00 | 0.00 | 0.00 |
| 1 | 24 | 0.00 | 0.00 | 0.00 | 0.00 |
| 2 | 48 | 0.00 | 0.00 | 0.00 | 0.00 |
| 3 | 72 | 0.00 | 0.00 | 0.00 | 0.00 |
| 5 | 120 | 0.17 | 0.10 | 0.07 | 0.16 |
| 7 | 168 | 0.11 | 0.18 | 0.12 | 0.20 |
| 15 | 360 | 0.00 | 0.00 | 0.18 | 0.00 |
| 22 | 528 | 0.00 | 0.00 | 0.09 | 0.00 |
| 29 | 696 | 0.00 | 0.00 | 0.26 | 0.00 |
| 36 | 864 | 0.00 | 0.00 | 0.26 | 0.00 |
| 43 | 1032 | 0.00 | 0.00 | 0.20 | 0.00 |
| 50 | 1200 | 0.00 | 0.00 | 0.17 | 0.00 |
| 57 | 1368 | 0.00 | 0.00 | 0.13 | 0.00 |
| 60 | 1440 | 0.00 | 0.00 | 0.07 | 0.00 |

TABLE 10
Group Mean Plasma Buprenorphine (ng/mL) × Time (hr): Combined Sexes

| | | 1068-57 | | 1068-60 | | 1068-78 | |
|---|---|---|---|---|---|---|---|
| | | Plasma | | Plasma | | Plasma | |
| Time | Time | BUP | SD | BUP | SD | BUP | SD |
| (day) | (hr) | (ng/mL) | (ng/mL) | (ng/mL) | (ng/mL) | (ng/mL) | (ng/mL) |
| 1 | 0 | 0.00 | 0.000 | 0.00 | 0.000 | 0.00 | 0.000 |
| 1 | 0.5 | 1.17 | 0.615 | 7.36 | 2.754 | 4.37 | 4.019 |
| 1 | 1 | 1.33 | 0.836 | 4.90 | 1.810 | 3.08 | 3.256 |
| 1 | 1.5 | 1.12 | 0.808 | 6.00 | 2.832 | 1.96 | 1.282 |
| 1 | 2 | 1.45 | 1.000 | 6.56 | 1.920 | 2.19 | 1.721 |
| 1 | 3 | 2.19 | 1.252 | 10.36 | 4.628 | 2.06 | 1.203 |
| 1 | 4 | 2.44 | 2.069 | 8.38 | 4.303 | 2.23 | 1.082 |
| 1 | 6 | 1.97 | 0.796 | 5.01 | 1.422 | 2.30 | 0.939 |
| 1 | 8 | 1.95 | 0.980 | 6.48 | 4.193 | 2.76 | 1.514 |
| 1 | 12 | 1.87 | 1.206 | 8.83 | 7.052 | 2.13 | 0.570 |
| 1 | 24 | 1.98 | 0.518 | 6.25 | 2.446 | 2.26 | 0.442 |
| 2 | 48 | 1.10 | 0.092 | 7.70 | 5.562 | 2.51 | 1.137 |
| 3 | 72 | 1.05 | 0.126 | 13.11 | 8.976 | 3.85 | 1.956 |
| 5 | 120 | 1.61 | 0.427 | 27.33 | 4.770 | 14.28 | 6.808 |
| 7 | 168 | 4.41 | 1.619 | 20.80 | 6.632 | 20.07 | 10.031 |
| 15 | 360 | 2.47 | 0.797 | 1.18 | 0.691 | 3.98 | 2.614 |
| 22 | 528 | 3.24 | 0.348 | 0.59 | 0.389 | 0.85 | 0.637 |
| 29 | 696 | 5.71 | 1.764 | 0.17 | 0.282 | 0.49 | 0.401 |
| 36 | 864 | 4.14 | 1.124 | 0.11 | 0.210 | 0.15 | 0.134 |
| 43 | 1032 | 1.64 | 1.019 | 0.07 | 0.146 | 0.05 | 0.072 |
| 50 | 1200 | 0.33 | 0.252 | 0.07 | 0.135 | 0.04 | 0.072 |
| 57 | 1368 | 0.03 | 0.033 | 0.04 | 0.089 | 0.03 | 0.065 |
| 60 | 1440 | 0.00 | 0 | 0.04 | 0.073 | 0.02 | 0.049 |

TABLE 11
Pharmacokinetic Parameters for Formulations 1068-57, 1068-60, and 1068-78

| | | Animal | t_{1/2} | Tₘₐₓ | Tₗₐₛₜ | Cₘₐₓ | AUCₗₐₛₜ | AUC_{0-INF} | Cl_F_{obs} |
|---|---|---|---|---|---|---|---|---|---|
| Group | Sex | ID | (hr) | (hr) | (hr) | (ng/mL) | (hr*ng/mL) | (hr*ng/mL) | (mL/hr/kg) |
| 1068-57 | | | | | | | | | |
| 1 | F | 7 | 79 | 696 | 1368 | 7.1 | 3766 | 3773 | 1590 |
| 1 | F | 8 | 57 | 696 | 1368 | 6.5 | 4300 | 4304 | 1394 |
| 1 | M | 1 | 68 | 168 | 1200 | 5.3 | 2613 | 2623 | 2288 |
| 1 | M | 2 | 74 | 696 | 1200 | 6.1 | 3703 | 3717 | 1614 |
| | | Mean | 70 | 564 | 1284 | 6.2 | 3595 | 3604 | 1722 |
| | | SD | 9 | 264 | 97 | 0.8 | 708 | 706 | 390 |
| 1068-60 | | | | | | | | | |
| 2 | F | 9 | 270 | 120 | 1440 | 25.0 | 4495 | 4552 | 1318 |
| 2 | F | 10 | 72 | 120 | 696 | 32.5 | 4780 | 4789 | 1253 |
| 2 | M | 3 | 59 | 168 | 528 | 29.8 | 4218 | 4257 | 1409 |
| 2 | M | 4 | 59 | 120 | 528 | 29.9 | 3438 | 3457 | 1735 |
| | | Mean | 115 | 132 | 798 | 29.3 | 4233 | 4264 | 1429 |
| | | SD | 103 | 24 | 435 | 3.1 | 577 | 580 | 214 |
| 1068-78 | | | | | | | | | |
| 3 | F | 11 | 186 | 168 | 1440 | 8.8 | 3255 | 3281 | 1829 |
| 3 | F | 12 | 90 | 168 | 864 | 32.5 | 5104 | 5115 | 1173 |
| 3 | M | 5 | 78 | 120 | 696 | 22.6 | 3276 | 3294 | 1821 |
| 3 | M | 6 | 142 | 168 | 1032 | 22.6 | 3471 | 3482 | 1723 |
| | | Mean | 124 | 156 | 1008 | 21.6 | 3776 | 3793 | 1637 |
| | | SD | 50 | 24 | 318 | 9.7 | 890 | 886 | 313 |

1. A method for treating opioid dependence and/or pain in a subject in need thereof, the method comprising administering to said subject a therapeutically effective amount of a sustained release buprenorphine microsphere (SRBM) formulation, wherein the SRBM formulation comprises: buprenorphine and poly(D,L-lactide-co-glycolide) having a MW of 24,000-38,000, wherein the SRBM formulation provides plasma concentration of buprenorphine in the subject that does not exceed 10 ng/ml in the 12 hours after administration.

2. The method of claim 1 wherein the formulation comprises:
buprenorphine and poly(D,L-lactide-co-glycolide) having a MW of 7,000-17,000.

3. The method of claim 1, wherein the SRBM formulation comprises buprenorphine and poly(D,L-lactide-co-glycolide) having a MW of 24,000-38,000 and poly(D,L-lactide-co-glycolide) having a MW of 7,000-17,000.

4. The method of claim 1, wherein the SRBM formulation further comprises polyvinyl alcohol or ethyl acetate.

5. (canceled)

6. (canceled)

7. The method of claim 1, wherein the pain is chronic pain in an opioid dependent subject.

8. The method of claim 1, wherein the plasma concentration of buprenorphine remains substantially within the therapeutic range for 29 or 30 days, and wherein the therapeutic range comprises from about 1 ng/mL to about 8 ng/mL for the treatment of opioid dependence or for the treatment of pain in opioid dependent subjects.

9.-12. (canceled)

13. The method of claim 1, wherein the SRBM formulation comprises from about 1 milligram to about 600 milligrams of buprenorphine.

14. The method of claim 1, wherein the buprenorphine is in the form of a free base or a pharmaceutically acceptable salt.

15. The method of claim 1, wherein the method comprises administering the SRBM formulation to the subject by injection once about every 7 days, once about every 30 days, once about every 60 days, once about every 120 days or once about every 180 days.

16.-19. (canceled)

20. The method of claim 1, wherein the SRBM formulation has a substantially linear release profile of buprenorphine that lasts for 30 days after administration to the subject.

21. The method of claim 1, wherein the SRBM formulation is administered a second time at least about 15 days, 30 days, 45 days, 60 days, 90 days, 120 days or 180 days after first administration.

22. The method of claim 1, wherein the SRBM formulation is administered by a subcutaneous injection or an intramuscular injection.

23. (canceled)

24. (canceled)

25. The method of claim 1, wherein the plasma concentration of buprenorphine in the subject within the first hour after administration is less than 10 ng/mL, or less than 7.5 ng/mL.

26. The method of claim 1, wherein the plasma concentration of buprenorphine in the subject within the first twelve hours after administration does not exceed about 10 ng/mL, 9 ng/mL, 8 ng/mL, 7.5 ng/mL, 7 ng/mL, 6.5 ng/mL, 6 ng/mL, 5.5 ng/mL or 5 ng/mL.

27.-34. (canceled)

35. The method of claim 1, wherein the plasma concentration of buprenorphine in the subject within the first twelve hours after administration does not exceed about 10 ng/mL or about 9 ng/mL.

36. (canceled)

37. The method of claim 1, wherein the plasma concentration of buprenorphine from 1 hour to 12 hours after administration of the SRBM formulation to the subject does not vary by more than 8 ng/mL (from the lowest to the highest concentration achieved).

38. The method of claim 1, wherein the plasma concentration of buprenorphine from 30 minutes to about 12 hours after administration of the SRBM formulation to the subject does not vary by more than 7.5 ng/mL.

39. The method of claim 1, wherein the plasma concentration of buprenorphine reaches and is maintained at or near a therapeutic level within 6 hours after administration of the SRBM formulation to the subject.

40. The method of claim 1, wherein the plasma concentration of buprenorphine in the subject remains near a therapeutic level from day 2 through day 30 after administration of the SRBM formulation to the subject.

41. (canceled)

42. The method of claim 40, wherein the therapeutic level comprises from about 1 ng/ml to about 8 ng/ml of buprenorphine for the treatment of opioid dependence, or for the treatment of pain in opioid dependent subjects.

43. (canceled)

44. (canceled)

45. The method of claim 1, wherein the plasma concentration of buprenorphine in the subject remains at a substantially therapeutic level from about day 2 through day 30, from about 12 hours through about day 35, or from about day 5 through about day 40 after administration of the SRBM formulation to the subject.

46. (canceled)

47. (canceled)

48. The method of claim 1, wherein the plasma concentration of buprenorphine in the subject remains at a substantially therapeutic level from about day 2 through about day 30, or about day 40, or about day 60, or about day 120, or about day 180 after administration of the SRBM formulation to the subject, or
wherein the plasma concentration of buprenorphine remains at a substantially therapeutic level from about 12 hours through about day 30, or about day 40, or about day 60, or about day 120, or about day 180 after administration of the SRBM formulation to the subject.

49. (canceled)

50. The method of claim 1, wherein the SRBM formulation provides Cₘₐₓ of burprenorphine that does not exceed 10 ng/mL, 9 ng/mL, 8 ng/mL, 7 ng/mL, 6.25 ng/mL, 6 ng/mL, 5.75 ng/mL, 5.5 ng/mL, 5.25 ng/mL, 5 ng/mL, 4.75 ng/mL, 4 ng/mL, 3 ng/mL, or 2 ng/mL.

51. The method of claim 1, further comprising administering a second or subsequent effective amount of a sustained release buprenorphine microsphere (SRBM) formulation, wherein the Cₘₐₓ of buprenorphine provided by the SRBM formulation after the second or subsequent administration is less than 10 ng/mL.

52. The method of claim 51, wherein the Cₘₐₓ of buprenorphine after the second or subsequent administration includes the residual amount from the first or previous administrations, and wherein the effective amount is between about 1 mg and about 600 mg.

53. (canceled)

54. The method of claim 1, wherein the SRBM formulation comprises particles having diameters between about 12 to about 100 μm, or between about 15 to about 80 μm, or between about 20 and about 40 μm.

55. (canceled)

56. (canceled)
